(19) United States
(12) Patent Application Publication
(10) Pub. No.: US 2021/0338713 A1
(43) Pub. Date: Nov. 4, 2021

(54) Reduction of Pulmonary Inflammation Using Therapeutic Gas Mixtures

(71) Applicant: Brain Cancer Research Institute, San Diego, CA (US)

(72) Inventors: Thomas Ichim, San Diego, CA (US); Feng Lin, San Diego, CA (US); Sandeep Pingle, San Diego, CA (US); Shashanka Ashili, Rosemount, MN (US)

(21) Appl. No.: 17/306,719

(22) Filed: May 3, 2021

Related U.S. Application Data
(60) Provisional application No. 63/019,878, filed on May 4, 2020.

(51) Int. Cl. A61K 33/00; A61K 9/00

(52) U.S. Cl. CPC A61K 33/00; A61K 9/0004

(57) ABSTRACT

Disclosed are compositions of matter, protocols, and combination therapies useful for reduction of pulmonary inflammation associated with conditions such as acute respiratory distress syndrome (ARDS), ventilator induced inflammation, and infectious disease induced inflammation. In one embodiment the invention provides therapeutic gases which induce anti-apoptotic, anti-inflammatory, and immune modulatory properties in the pulmonary environment of a patient in need of therapy.

### CROSS-REFERENCE TO RELATED APPLICATIONS

This application claims priority to U.S. Provisional Application No. 63/019,878, titled “Reduction of Pulmonary Inflammation Using Therapeutic Gas Mixtures”, filed May 4, 2020, which is incorporated by reference in its entirety.

### FIELD OF THE INVENTION

The invention pertains to the field of SARS-CoV-2 therapeutics, more specifically, the treatment of SARS-CoV-2 induced neurological and pulmonary inflammation such as ARDS.

### BACKGROUND

Acute Respiratory Distress Syndrome (ARDS) is a condition of acute respiratory failure caused by a variety of factors which are related to inflammation and release of various activators of the innate immune system such as cytokines and pyrogenic factors [1-9].

It is widely known that ARDS generally presents with progressive hypoxemia, dyspnea and increased work of breathing. Patients often require mechanical ventilation and supplemental oxygen [10]. Over the years, our understanding of ARDS has advanced significantly, with elucidation of several of the molecular and cellular pathways involved in initiation, progression and resolution/fibrosis. However, ARDS is still represents significant morbidity and mortality and therapeutic strategies to mitigate the foregoing have resulted in limited translational success. Part of this failure stems from the very different presentations of ARDS between people, as well as differences in their genetic composition.

ARDS is caused by many situations bacterial and viral pneumonia, sepsis, inhalation of harmful substances, head, chest or other major injury, burns, blood transfusions, near drowning, aspiration of gastric contents, pancreatitis, intravenous drug use, and abdominal trauma. Furthermore, those with a history of chronic alcoholism are at a higher risk of developing ARDS [11-13]. Alcoholism affects several parameters relevant to ARDS including: a) reduction in glutathione levels [14, 15], b) increasing levels of adhesion molecules on lung blood vessels so as to increase recruitment of inflammatory cells [16]; c) upregulating lung adenosine levels, resulting in impaired active Na(+) transport in the lung [17]; and d) suppression of pulmonary immunity [18].

One of the cardinal symptoms of ARDS is fluid accumulation in the lungs. When this occurs, the elastic air sacs (alveoli) in the lungs fill with fluid and the function of the alveoli is impaired. The result is that less oxygen reaches the bloodstream, depriving organs of the oxygen required for normal function and viability. In some instances, ARDS occurs in people who are already critically ill or who have significant injuries. Severe shortness of breath, the main symptom of ARDS, usually develops within a few hours to a few days after the precipitating injury or infection.

Unfortunately, many patients who develop ARDS do not survive. The risk of death increases with age and severity of illness. Of the people who do survive ARDS, some recover completely while others experience lasting damage to their lungs.

Currently there exist no effective pharmacologic therapies for treatment or prevention of ARDS. While inhibition of fibrin formation mitigated injury in some preclinical models of ARDS, anticoagulation therapies in humans do not attenuate ARDS and may even increase mortality. Protective lung ventilator strategies remain the mainstay of available treatment options. Due to the significant morbidity and mortality associated with ARDS and the lack of effective treatment options, new therapeutic agents for the treatment of ARDS and new treatment methods for ARDS are needed.

The present disclosure addresses the unmet need in the art by providing novel therapeutic agents useful in the treatment of ARDS and methods of treatment for ARDS and conditions related thereto through the administration of such novel therapeutic agents.

### SUMMARY

Various aspects of the invention are enumerated in the following paragraphs.

Preferred embodiments are directed to methods of treating pulmonary inflammation comprising administering to a patient in need of therapy a therapeutic gas composition at a concentration and frequency needed to reduce said pulmonary inflammation.

Preferred embodiments are directed to methods wherein said pulmonary inflammation is acute respiratory distress syndrome.

Preferred embodiments are directed to methods wherein said pulmonary inflammation is enhanced levels of inflammatory cytokines in the lung as compared to an age-matched control lung.

Preferred embodiments are directed to methods wherein said cytokines in the lung are assessed by bronchoalveolar lavage.

Preferred embodiments are directed to methods wherein said inflammatory cytokines are selected from a group comprising of: a) interleukin 1; b) interleukin 6; c) interleukin 8; d) interleukin-11; e) interleukin 12; f) interleukin 15; g) interleukin 17; h) interleukin 18; i) interleukin 33; j) TNF-alpha; k) HMGB1; and 1) MCP-1.

Preferred embodiments are directed to methods wherein said pulmonary inflammation is enhanced numbers of neutrophils in the lung as compared to an age-matched control lung.

Preferred embodiments are directed to methods wherein said pulmonary inflammation is enhanced numbers of activated in the lung as compared to an age-matched control lung.

Preferred embodiments are directed to methods wherein said pulmonary inflammation is reduced propensity of neutrophils to undergo apoptosis in the lung as compared to an age-matched control lung.

Preferred embodiments are directed to methods wherein said pulmonary inflammation is enhanced numbers of Th17 cells in the lung as compared to an age-matched control lung.

Preferred embodiments are directed to methods wherein said pulmonary inflammation is reduced numbers of Treg cells in the lung as compared to an age-matched control lung.

Preferred embodiments are directed to methods wherein said Th17 cells express ROR gamma T

Preferred embodiments are directed to methods wherein said Treg cells express FoxP3.

Preferred embodiments are directed to methods wherein said pulmonary inflammation is an increased number of neutrophil extracellular traps in the lung as compared to an age-matched control lung.

Preferred embodiments are directed to methods wherein said therapeutic gases comprise of a mixture of one or more gases: a) carbon monoxide; b) argon; c) helium; c) radon; d) krypton; e) neon; f) xenon; g) hydrogen; h) hydrogen sulfide; i) ozone and j) nitric oxide.

Preferred embodiments are directed to methods wherein said therapeutic gases comprise a proportion by volume of 20 to 70% of therapeutic gas and the rest oxygen and/or air.

Preferred embodiments are directed to methods wherein said proportion of xenon is between 22 and 60% by volume to oxygen.

Preferred embodiments are directed to methods wherein said proportion of xenon is between 25 and 60% by volume to oxygen.

Preferred embodiments are directed to methods wherein said therapeutic gas consists of a) oxygen and xenon or b) air and xenon.

Preferred embodiments are directed to methods wherein said therapeutic gas mixture also contains nitrogen, helium, Nitric Oxide, krypton, argon, hydrogen sulfide or neon.

Preferred embodiments are directed to methods wherein said therapeutic gas mixture contains a proportion by volume of oxygen of between 15 and 25%.

Preferred embodiments are directed to methods wherein said therapeutic gas mixture is supplied for inhalation from a pressurized container at a pressure greater than 2 bar.

Preferred embodiments are directed to methods wherein said therapeutic gas mixture is administered intranasally.

Preferred embodiments are directed to methods wherein said therapeutic gas mixture is administered through the use of a hyperbaric chamber.

Preferred embodiments are directed to methods wherein said hyperbaric chamber is pressurized to a pressure of no more than 3 atm (0.3 MPa).

Preferred embodiments are directed to methods wherein a therapeutic gas mixture is administered to the patient while the patient is in the hyperbaric environment.

Preferred embodiments are directed to methods wherein said therapeutic gas mixture is administered by inhalation or simulated inhalation.

Preferred embodiments are directed to methods wherein said therapeutic gas mixture is xenon, helium, or a mixture of xenon and helium.

Preferred embodiments are directed to methods wherein the therapeutic gas mixture is xenon or a mixture of xenon and helium, and the partial pressure of xenon is no more than about 0.8 atm (0.08 MPa).

Preferred embodiments are directed to methods wherein said therapeutic gas mixture is administered mixed with air, the air partial pressure being about 1 atm (0.1 MPa).

Preferred embodiments are directed to methods wherein said therapeutic gas mixture is administered as part of a gas mixture comprising oxygen, the nitrogen partial pressure in the mixture being equal to or less than about 0.8 atm (0.08 MPa).

Preferred embodiments are directed to methods wherein said gas mixture is essentially free of nitrogen.

Preferred embodiments are directed to methods wherein the oxygen partial pressure is about 0.2 atm (0.02 MPa).

Preferred embodiments are directed to methods wherein said therapeutic gas mixture contains nitric oxide at a concentration of 5-50 parts per million.

Preferred embodiments are directed to methods wherein said therapeutic gas mixture contains ozone gas at a concentration of 1-200 parts per million.

Preferred embodiments are directed to methods wherein said therapeutic gas mixture contains hydrogen gas at a concentration of 1-1000 parts per million.

Preferred embodiments are directed to methods wherein said therapeutic gas mixture contains carbon monoxide gas at a concentration of 0.01-10 parts per million.

Preferred embodiments are directed to methods wherein said therapeutic gas mixture contains hydrogen sulfide gas at a concentration of 1-10,000 parts per million.

Preferred embodiments are directed to methods wherein said therapeutic gas mixture contains neon gas at a concentration of 1-20% volume by volume.

Preferred embodiments are directed to methods wherein said therapeutic gas mixture contains argon gas at a concentration of 1-20% volume by volume.

Preferred embodiments are directed to methods wherein said therapeutic gas mixture contains krypton gas at a concentration of 1-20% volume by volume.

Preferred embodiments are directed to methods wherein said therapeutic gas mixture contains radon gas at a concentration of 1-10,000 parts per million.

Preferred embodiments are directed to methods wherein said administration of therapeutic gases protects the lung from ventilator induced inflammation.

Preferred embodiments are directed to methods wherein said administration of therapeutic gases induces an anti-apoptotic activity in pulmonary epithelial cells.

Preferred embodiments are directed to methods wherein said administration of therapeutic gases induces enhanced expression of KGF-1.

Preferred embodiments are directed to methods of treating neurological inflammation associated with COVID-19 comprising administering to a patient in need of therapy a therapeutic gas composition at a concentration and frequency needed to reduce said brain inflammation.

Preferred embodiments are directed to methods wherein said neurological inflammation is associated with increased microglial activation.

Preferred embodiments are directed to methods wherein said neurological inflammation is associated with increased production of TNF-alpha.

Preferred embodiments are directed to methods wherein said neurological inflammation is associated with increased production of IL-1 beta.

Preferred embodiments are directed to methods wherein said neurological inflammation is associated with increased production of IL-6.

Preferred embodiments are directed to methods wherein said neurological inflammation is associated with increased production of IL-8.

Preferred embodiments are directed to methods wherein said neurological inflammation is associated with increased production of IL-17.

Preferred embodiments are directed to methods wherein said neurological inflammation is associated with increased production of IL-23.

Preferred embodiments are directed to methods wherein said cytokines are elevated in the peripheral blood.

Preferred embodiments are directed to methods wherein said cytokines are elevated in the cerebral spinal fluid.

Preferred embodiments are directed to methods wherein said neurological inflammation is associated with activation of indolamine-2,3-deoxygenase.

Preferred embodiments are directed to methods wherein said neurological inflammation is associated with activation of glutamatergic signaling.

Preferred embodiments are directed to methods wherein said neurological inflammation is enhanced numbers of T cells into the brain as compared to an age-matched control brain.

Preferred embodiments are directed to methods wherein said neurological inflammation is enhanced numbers of activated T cells in the brain as compared to an age-matched control brain.

Preferred embodiments are directed to methods wherein said neurological inflammation is reduced propensity of T cells to undergo apoptosis in the CNS as compared to an age-matched control CNS.

Preferred embodiments are directed to methods wherein said neurological inflammation is enhanced numbers of Th17 cells in the brain as compared to an age-matched control brain.

Preferred embodiments are directed to methods wherein said neurological inflammation is reduced numbers of Treg cells in the brain as compared to an age-matched control brain.

Preferred embodiments are directed to methods wherein said Th17 cells express ROR gamma T

Preferred embodiments are directed to methods wherein said Treg cells express FoxP3.

Preferred embodiments are directed to methods wherein said brain inflammation is an increased number of extracellular TLR agonists in the brain as compared to an age-matched control brain.

Preferred embodiments are directed to methods wherein said therapeutic gases comprise of a mixture of one or more gases: a) carbon monoxide; b) argon; c) helium; c) radon; d) krypton; e) neon; f) xenon; g) hydrogen; h) hydrogen sulfide; i) ozone and j) nitric oxide.

Preferred embodiments are directed to methods wherein said therapeutic gases comprise a proportion by volume of 20 to 70% of therapeutic gas and the rest oxygen and/or air.

Preferred embodiments are directed to methods wherein said proportion of xenon is between 22 and 60% by volume to oxygen.

Preferred embodiments are directed to methods wherein said proportion of xenon is between 25 and 60% by volume to oxygen.

Preferred embodiments are directed to methods wherein said therapeutic gas consists of a) oxygen and xenon or b) air and xenon.

Preferred embodiments are directed to methods wherein said therapeutic gas mixture also contains nitrogen, helium, Nitric Oxide, krypton, argon, hydrogen sulfide or neon.

Preferred embodiments are directed to methods wherein said therapeutic gas mixture contains a proportion by volume of oxygen of between 15 and 25%.

Preferred embodiments are directed to methods wherein said therapeutic gas mixture is supplied for inhalation from a pressurized container at a pressure greater than 2 bar.

Preferred embodiments are directed to methods wherein said therapeutic gas mixture is administered intranasally.

Preferred embodiments are directed to methods wherein said therapeutic gas mixture is administered through the use of a hyperbaric chamber.

Preferred embodiments are directed to methods wherein said hyperbaric chamber is pressurized to a pressure of no more than 3 atm (0.3 MPa).

Preferred embodiments are directed to methods wherein a therapeutic gas mixture is administered to the patient while the patient is in the hyperbaric environment.

Preferred embodiments are directed to methods wherein said therapeutic gas mixture is administered by inhalation or simulated inhalation.

Preferred embodiments are directed to methods wherein said therapeutic gas mixture is xenon, helium, or a mixture of xenon and helium.

Preferred embodiments are directed to methods wherein the therapeutic gas mixture is xenon or a mixture of xenon and helium, and the partial pressure of xenon is no more than about 0.8 atm (0.08 MPa).

Preferred embodiments are directed to methods wherein said therapeutic gas mixture is administered mixed with air, the air partial pressure being about 1 atm (0.1 MPa).

Preferred embodiments are directed to methods wherein said therapeutic gas mixture is administered as part of a gas mixture comprising oxygen, the nitrogen partial pressure in the mixture being equal to or less than about 0.8 atm (0.08 MPa).

Preferred embodiments are directed to methods wherein said gas mixture is essentially free of nitrogen.

Preferred embodiments are directed to methods wherein the oxygen partial pressure is about 0.2 atm (0.02 MPa).

Preferred embodiments are directed to methods wherein said therapeutic gas mixture contains nitric oxide at a concentration of 5-50 parts per million.

Preferred embodiments are directed to methods wherein said therapeutic gas mixture contains ozone gas at a concentration of 1-200 parts per million.

Preferred embodiments are directed to methods wherein said therapeutic gas mixture contains hydrogen gas at a concentration of 1-1000 parts per million.

Preferred embodiments are directed to methods wherein said therapeutic gas mixture contains carbon monoxide gas at a concentration of 0.01-10 parts per million.

Preferred embodiments are directed to methods wherein said therapeutic gas mixture contains hydrogen sulfide gas at a concentration of 1-10,000 parts per million.

Preferred embodiments are directed to methods wherein said therapeutic gas mixture contains neon gas at a concentration of 1-20% volume by volume.

Preferred embodiments are directed to methods wherein said therapeutic gas mixture contains argon gas at a concentration of 1-20% volume by volume.

Preferred embodiments are directed to methods wherein said therapeutic gas mixture contains krypton gas at a concentration of 1-20% volume by volume.

Preferred embodiments are directed to methods wherein said therapeutic gas mixture contains radon gas at a concentration of 1-10,000 parts per million.

Preferred embodiments are directed to methods wherein said administration of therapeutic gases protects the brain from ventilator induced inflammation.

Preferred embodiments are directed to methods wherein said administration of therapeutic gases induces an anti-apoptotic activity in neural cells.

Preferred embodiments are directed to methods wherein said administration of therapeutic gases induces enhanced expression of neurotrophic cytokines.

Preferred embodiments are directed to methods wherein said neurotrophic cytokines are selected from a group comprising of: a) BDNF; b) NGF; c) CTNF; and d) a member of the FGF family.

Preferred embodiments are directed to methods wherein said therapeutic gases are administered together with a cellular therapy.

Preferred embodiments are directed to methods wherein said cellular therapy is selected from a group of therapies comprising of: a) mesenchymal stem cells; b) hematopoietic stem cells; and c) immune cells.

Preferred embodiments are directed to methods wherein said cells are treated with said gas-based therapies before administration in vivo.

Preferred embodiments are directed to methods wherein said gas based therapy enhances regenerative activity of said cell therapy.

Preferred embodiments are directed to methods in which said regenerative activity is ability to secrete growth factors.

Preferred embodiments are directed to methods in which growth factors are selected from a group comprising of: a) HGF-1; b) FGF-1; c) FGF-2; d) FGF-5; e) IGF-1; f) EGF; g) PDGF=BB; h) angiopoietin; and i) interleukin-35.

Preferred embodiments are directed to methods wherein said regenerative activity is production of angiogenic factors.

Preferred embodiments are directed to methods wherein said angiogenic factors are selected from a group comprising of: a) VEGF; b) angiopoietin; and c) placental angiogenic factor.

Preferred embodiments are directed to methods wherein said regenerative activity is ability to differentiate into injured tissue.

Preferred embodiments are directed to methods wherein said regenerative activity is ability to transfer mitochondria from said regenerative cell into cells which comprise injured tissue.

### DETAILED DESCRIPTION OF THE INVENTION

The invention teaches means of utilizing various therapeutic gases for treatment of SARS-CoV-2 induced pathology, including neurological and pulmonary inflammation such as ARDS.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein and includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. In another embodiment, the subject is a domesticated animal including companion animals (e.g., dogs, cats, rats, guinea pigs, hamsters etc.).

The term “chronic administration” means the administration of an agent (e.g., a xenon composition as described herein) on a periodic basis (e.g., at least once a day, twice a day, three times a day, four times a day, at least once a week, twice a week, three times a week, four times a week, five times a week, six times a week, seven times a week, once a month, twice a month, three times a month, and four times a month) over an extended period of time (e.g., at least one month, two months, three months, four months, five months, six months, seven months, eight months, nine months, ten months, eleven months, one year, two years, three years, four years, five years, six years, seven years, eight years, nine years, or ten years). The periodic administration of the agent (e.g., a gaseous composition such as a xenon composition as described herein) can be performed over a continuous period of time (as described herein) or can be administered as a bolus (e.g., inhalation of a single dose of gas or administration of a dosage of a gas based composition (e.g., a nanoparticle or nanosponge).

The phrase “continuous period of time” means at least 5 minutes, (e.g., at least 10 minutes, at least 15 minutes, 30 minutes, 45 minutes, 1 hour, 2 hours, 3 hours, 4 hours, 5 hours, 6 hours, 7 hours, 8 hours, 9 hours, 10 hours, 11 hours, or 12 hours, overnight, or between 5 and 13 hours).

As used herein, “acute” administration of a the therapeutic gas composition means a single exposure within an extended time period of the subject to the therapeutically effective amount of xenon. In conjunction with this definition of “acute”, an extended time period is defined as four days or longer, e.g., once-weekly administration of said therapeutic gas composition constitutes acute administration. Administering a dose of a xenon composition to a subject, followed by a second dose 24 hours later, does not constitute acute dosing. Repeated administration or self-administration to achieve a desired effect by administering at least a second exposure can still be considered “acute” administration when repeated dosing is required to titrate a dose to reduce the intensity

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “pharmaceutically acceptable” can refer to compounds and compositions which can be administered to a subject (e.g., a mammal or a human) without undue toxicity.

As used herein, the term “pharmaceutically acceptable carrier” can include any material or substance that, when combined with an active ingredient allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. The term “pharmaceutically acceptable carriers” excludes tissue culture media. For the purpose of the invention, pharmaceutical carriers may include various types of nanoparticles, sponges, or even cells.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

It should be understood that this invention is not limited to the particular methodologies, protocols, and reagents, etc., described herein and as such can vary therefrom. The terminology used herein is for the purpose of describing particular embodiments only.

The term “xenon” as used herein is not intended to restrict the present invention to a gas or liquid of pure xenon. The term also encompasses a composition comprising xenon--such as a mixture of xenon and oxygen. Nevertheless, when xenon is used as the sole organ and/or tissue and/or cell protectant then no agent (such as carbon monoxide) may be added to a mixture at a dosage wherein said agent is capable of acting as an organ and/or tissue and/or cell protectant. Preferably said agent is not capable of acting as an organ and/or tissue and/or cell protectant at any dosage. Xenon (Xe) is an atom (atomic number 54) existing in the ambient atmosphere in low concentration (0.0000086% or 0.086 part per million (ppm) or 86 parts per million (ppb)). When purified it is presented as a gas in normobaric situations. Xenon is one of the inert or “Nobel” gases including also argon and Krypton. Due to its physiochemical properties xenon gas is heavier then normal air, with a specific gravity or density of 5.887 g/1 and its oil/gas partition coefficient is 1.9 and “blood/gas” partition coefficient of about 0.14. In concentrations of 10-70 vol. % in combination with oxygen, xenon exhibits anaesthetic effects. A number of studies in humans have looked at the effects of both hyperbaric and normobaric effects of xenon and shown dose dependent analgesic properties similar to those of nitrous oxide and that xenon in higher concentration exhibits anaesthetic properties and creates a drug induced stage of sleep and depression of response to painful stimuli. For the purpose of the invention, the uptake of xenon or other therapeutic gases can occur via the respiratory system and the transport into the brain are already known from the use of xenon as an anaesthetic agent. It can also be assumed, from its use as anaesthetic agent, that the use of xenon has no damaging effect on an organism. Moreover, studies have shown that xenon exposure does not induce significant toxic effects on main organs. Helium may be added to xenon gas since helium is a molecule of small size it may function as carrier for the more voluminous xenon. Furthermore, further gases having medical effects may be added to the xenon composition, e.g. NO or CO.sub.2. In addition, depending on the disease to be treated other medicaments which are preferably inhalable may be added, e.g. cortisons, antibiotics etc. However when xenon is used as the sole organ and/or tissue and/or cell protectant then no other agent (such as carbon monoxide) may be added at a dosage wherein said agent is capable of acting as an organ and/or tissue and/or cell protectant. Preferably said agent is not capable of acting as an organ and/or tissue and/or cell protectant at any dosage.Xenon can be administered to an organ and/or tissue and/or cell as a xenon-saturated solution. One way in which a xenon-saturated solution may be prepared is to expose a buffered physiologic salt solution to 100% xenon, or alternatively 80% xenon/20% oxygen, in an air-tight plastic bag and mix for one hour on a shaker. The gas atmosphere is changed at least once and the mixing procedure repeated. Then a complete saturation of the buffer with the gas (mixture) is achieved.

For the practice of the invention, a xenon-saturated solution is particularly useful for transplantation and implantation purposes. If the organ and/or tissue and/or cell is maintained during transport or during the pre-operation phase in such a solution, a considerable reduction of the rate of apoptosis in the organ and/or tissue and/or cell can be observed.

In one embodiment, pulmonary inflammation is associated with enhance neutrophil extracellular traps being released and administration of the DNA cleaving drug dronase is provided as a first treatment to reduce inflammation, which will allow for enhanced efficacy of the second treatment which is gas-based.

In some embodiments, said gas-based treatments are utilized as monotherapy, in other embodiments, gas based therapies are used as adjuvants to existing therapies for SARS-CoV-2 induced disease such as COVID19. Existing therapies that are useful for combination include: hydroxychloroquine [19] alone or with zinc [20], Favipiravir [21], Lopinavir/Ritonavir [22, 23], Nucleoside analogues, Neuraminidase inhibitors, Remdesivir, peptide (EK1), abidol [24], RNA synthesis inhibitors (such as TDF, 3TC), anti-inflammatory drugs (such as hormones and other molecules), Chinese traditional medicine, such ShuFengJieDu Capsules and Lianhuaqingwen Capsule, extracorporeal membrane oxygenation [25], anti-complement C5 therapy with eculizumab [26],

In one embodiment a mixture of xenon, hydrogen, and nitric oxide is provided, wherein said mixture provides for reduction of inflammation and therapeutic synergies. ARDS is associated with activation of innate immune responses, locally and/or systemically. Various innate immune cells have been shown to participate in generation of the cytokine storm and/or other inflammatory means which culminate in water entering the alveoli and reducing ability of the lung to uptake oxygen and release carbon dioxide. For example, activation of pulmonary macrophages has been shown to be associated with generation of pulmonary inflammation and ARDS. One early study showed that macrophage activation, as detected by production of the inflammatory cytokine interleukin-1, is found in clinical cases. Investigators showed that In vitro, in ARDS patients, the alveolar macrophages released significantly more total IL-1 and IL-1 beta than patients with pneumonia and in control subjects. Importantly, after stimulation of alveolar macrophages with 10 micrograms/ml of lipopolysaccharide (LPS), only macrophages from ARDS patients stimulated enhanced production of IL-1. Incubation of AM with 250 U/ml human interferon-gamma (gamma IFN) was associated with less IL-1 beta release. However, stimulating AM from patients with ARDS and severe pneumonia with gamma IFN plus LPS enhanced the release of IL-1 beta compared with that in patients with pneumonia and in control subjects. These data suggest that in ARDS macrophages are constitutively active producing inflammatory mediators, and additionally, the macrophages are primed so as to become even more inflammatory after activation [27]. The role of macrophages as a culprit of initiating ARDS has been documented by others [28]. Signs of pulmonary macrophage activation are observed in ARDS patients including: a) NF-kappa B activation [29-32]; b) enhanced migration towards chemokines [33]; c) production of the neutrophil chemokine IL-8; d) MAPK/ERK activation [34]; e) stimulation of alveolar barrier dysfunction [35]; f) produce IL-17 g) HMGB-1 [36]; and h) in some situations macrophages in ARDS undergo a peculiar type of cell death called pyroptosis in which they release stimulators of inflammation and innate immunity [37].

The invention provides that various gases, in one preferred embodiment, Nobel Gases, possess ability to reduce inflammatory mediators associated with ARDS.

The utilization of nitric oxide in treatment of ARDS has been previously disclosed and publications are incorporated by reference. In an ovine model of ARDS inhalation of nitric oxide was shown to decrease pulmonary arterial pressure (PAP) and resistance without any systemic hemodynamic effects, increase arterial PO2, and decrease venous admixture (Qva/QT; all P<0.05) without altering cardiac output (QT), mixed venous PO2, or O2 uptake, major determinants of intrapulmonary shunt. During NO inhalation, PAP-left atrial pressure gradient (PAP-LAP) and Qva/QT were reduced (both P<0.05) independently of QT, which was varied mechanically [38]. In a pediatric patient ARDS trial, inhaled nitric oxide improved oxygenation in 15 of 17 patients, lowered mean pulmonary artery pressure and intrapulmonary shunt without changing systemic arterial pressure or pulmonary capillary wedge pressure. Fifteen patients treated with low-dose inhaled NO (3 to 10 ppm) for 1 to 24 days; 5 (50%) of 10 patients with ARDS and 7 (100%) of the 7 non-ARDS patients survived [39].

Activities of inhaled nitric oxide include inhibition of platelet aggregation [40, 41], improving oxygenation [42-53], reducing pulmonary hypertension [54, 55], reducing adhesion molecules on endothelial cells such as ICAM-1 [56], reduction of TLR2 and TLR4 [57], increasing endothelial permeability [58], stimulation of endothelial progenitor cell mobilization [59],

Other signals of efficacy have been reported for nitric oxide, for example, RDS patients surviving after treatment with low-dose iNO had significantly better values for select pulmonary function tests at six months post-treatment than placebo-treated patients [60].

Unfortunately, nitric oxide has not been clinically approved by regulators, in part because of lack of conclusive Phase III double blind studies being successful [60-62]. In some embodiments of the invention, therapeutic gases are used to reduce neurological manifestations of COVID-19, this is based on ability of gases such as xenon to cross blood brain barrier and excellent safety profile [63, 64], to inhibit neuroinflammation in animal models of stroke and ischemia/reperfusion [65-67], to possess clinical safety and efficacy in neonatal ischemia reperfusion [68], and to suppress NF-kappa B, TNF-alpha and inflammatory cytokines [69].

The therapeutically effective dose of a xenon and/or other therapeutic gas based composition can be administered using any medically acceptable mode of administration. Although the skilled artisan would contemplate any of the modes of administration known to one of ordinary skill, preferably xenon is administered by inhalation so that the effect will have a rapid onset and can be easily and rapidly titrated for appropriate treatment.

Therapeutic compositions of the agents disclosed herein can include a physiologically tolerable carrier together with xenon gas as described herein, dissolved or dispersed therein as an active ingredient. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without toxicity or the production of undesirable physiological effects such as nausea, dizziness, gastric upset and the like. A pharmaceutically acceptable carrier will not itself promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as topical agents or injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof In addition, if desired, the composition can contain minor amounts of auxiliary substances such as wetting or emulsifying agents, pH buffering agents and the like which enhance the effectiveness of the active ingredient. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

In some embodiments, it can be advantageous to formulate the aforementioned pharmaceutical compositions in dosage unit form for ease of administration and uniformity of dosage. Dosage unit or unitary form refers to physically discrete units suitable as unitary dosages (e.g., a metered inhaled dose), each unit containing a predetermined quantity of xenon gas calculated to produce the desired therapeutic effect in association with the required pharmaceutical carrier.

In some embodiments, stem cells are utilized together with therapeutic gases. In one particular embodiment, stem cells are stimulated using gas mixtures to enhance therapeutic effects. Mesenchymal stem cells are utilized in one preferred embodiment of the invention, and activated with gases before administration, and/or co-administered together with therapeutic gases. Means of generating MSC are known in the art and described below.

Mesenchymal stem cells (“MSC”) were originally derived from the embryonal mesoderm and subsequently have been isolated from adult bone marrow and other adult tissues. They can be differentiated to form muscle, bone, cartilage, fat, marrow stroma, and tendon. Mesoderm also differentiates into visceral mesoderm which can give rise to cardiac muscle, smooth muscle, or blood islands consisting of endothelium and hematopoietic progenitor cells. The differentiation potential of the mesenchymal stem cells that have been described thus far is limited to cells of mesenchymal origin, including the best characterized mesenchymal stem cell (See Pittenger, et al. Science (1999) 284: 143-147 and U.S. Pat. No. 5,827,740 (SH2.sup.+ SH4.sup.+ CD29.sup.+ CD44.sup.+ CD71.sup.+ CD90.sup.+ CD106.sup.+ CD120a.sup.+ CD124.sup.+ CD14.sup.− CD34.sup.− CD45.sup.−)). The invention teaches the use of various mesenchymal stem cells

In one embodiment MSC donor lots are generated from umbilical cord tissue. Means of generating umbilical cord tissue MSC have been previously published and are incorporated by reference [70-76]. The term “umbilical tissue derived cells (UTC)” refers, for example, to cells as described in U.S. Pat. Nos. 7,510,873, 7,413,734, 7,524,489, and U.S. Pat. No. 7,560,276. The UTC can be of any mammalian origin e.g. human, rat, primate, porcine and the like. In one embodiment of the invention, the UTC are derived from human umbilicus. umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle). In addition, these isolated human umbilicus-derived cells express a gene for each of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes.

Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activites selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activites is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

In one embodiment, bone marrow MSC lots are generated, means of generating BM MSC are known in the literature and examples are incorporated by reference.

In one embodiment BM-MSC are generated as follows
1. 500 mL Isolation Buffer is prepared (PBS+2% FBS+2 mM EDTA) using sterile components or filtering Isolation Buffer through a 0.2 micron filter. Once made, the Isolation Buffer was stored at 2-8.degree. C.
2. The total number of nucleated cells in the BM sample is counted by taking 10.mu.L BM and diluting it 1/50-1/100 with 3% Acetic Acid with Methylene Blue (STEMCELL Catalog #07060). Cells are counted using a hemacytometer.
3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of 70 mL).
4. In three 50 mL conical tubes (BD Catalog #352070), 17 mL Ficoll-Paque.™. PLUS (Catalog #07907/07957) is pipetted into each tube. About 23 mL of the diluted BM from step 3 was carefully layered on top of the Ficoll-Paque.™. PLUS in each tube.
5. The tubes are centrifuged at room temperature (15-25.degree. C.) for 30 minutes at 300.times.g in a bench top centrifuge with the brake off
6. The upper plasma layer is removed and discarded without disturbing the plasma:Ficoll-Paque.™. PLUS interface. The mononuclear cells located at the interface layer are carefully removed and placed in a new 50 mL conical tube. Mononuclear cells are resuspended with 40 mL cold (2-8.degree. C.) Isolation Buffer and mixed gently by pipetting.
7. Cells were centrifuged at 300.times.g for 10 minutes at room temperature in a bench top centrifuge with the brake on. The supernatant is removed and the cell pellet resuspended in 1-2 mL cold Isolation Buffer.
8. Cells were diluted 1/50 in 3% Acetic Acid with Methylene Blue and the total number of nucleated cells counted using a hemacytometer.
9. Cells are diluted in Complete Human MesenCult.™.-Proliferation medium (STEMCELL catalog #05411) at a final concentration of 1.times.10.sup.6 cells/mL.
10. BM-derived cells were ready for expansion and CFU-F assays in the presence of GW2580, which can then be used for specific applications.

Said BM-MSC are treated with Noble gas containing mixtures at a concentration and frequency sufficient to enhance HIF-1 alpha activity, which is used as a marker of augmented activity.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2 10⁷ cells/ml.

Subsequently the cells are centrifuged at 900 g for approximately 30 min or a time period sufficient to achieve separation of mononuclear cells from debris and erythrocytes. Said cells are then washed with PBS and plated at a density of approximately 1 10⁶ cells per ml in 175 cm² tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1 10⁶ per 175 cm². Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In some embodiments of the invention MSC are transferred to possess enhanced neuromodulatory and neuroprotective properties. Said transfection may be accomplished by use of lentiviral vectors, said means to perform lentiviral mediated transfection are well-known in the art and discussed in the following references [77-83]. Some specific examples of lentiviral based transfection of genes into MSC include transfection of SDF-1 to promote stem cell homing, particularly hematopoietic stem cells [84], GDNF to treat Parkinson's in an animal model [85], HGF to accelerate remyelination in a brain injury model [86], akt to protect against pathological cardiac remodeling and cardiomyocyte death [87], TRAIL to induce apoptosis of tumor cells [88-91], PGE-1 synthase for cardioprotection [92], NUR77 to enhance migration [93], BDNF to reduce ocular nerve damage in response to hypertension [94], HIF-1 alpha to stimulate osteogenesis [95], dominant negative CCL2 to reduce lung fibrosis [96], interferon beta to reduce tumor progression [97], HLA-G to enhance immune suppressive activity [98], hTERT to induce differentiation along the hepatocyte lineage [99], cytosine deaminase [100], OCT-4 to reduce senescence [101, 102], BAMBI to reduce TGF expression and protumor effects [103], HO-1 for radioprotection [104], LIGHT to induce antitumor activity [105], miR-126 to enhance angiogenesis [106, 107], bc1-2 to induce generation of nucleus pulposus cells [108], telomerase to induce neurogenesis [109], CXCR4 to accelerate hematopoietic recovery [110] and reduce unwanted immunity [111], wnt11 to promote regenerative cytokine production [112], and the HGF antagonist NK4 to reduce cancer [113].

Cell cultures are tested for sterility weekly, endotoxin by limulus amebocyte lysate test, and mycoplasma by DNA-fluorochrome stain.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington D.C., USA). MSCs harvested from each 10-flask set were stored for up to 4 h at 4° C. and combined at the end of the harvest. A total of 2-10 10⁶ MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, UT, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment of the invention enhanced MSC are transfected with anti-apoptotic proteins to enhance in vivo longevity. The present invention includes a method of using MSC that have been cultured under conditions to express increased amounts of at least one anti-apoptotic protein as a therapy to inhibit or prevent apoptosis. In one embodiment, the MSC which are used as a therapy to inhibit or prevent apoptosis have been contacted with an apoptotic cell. The invention is based on the discovery that MSC that have been contacted with an apoptotic cell express high levels of anti-apoptotic molecules. In some instances, the MSC that have been contacted with an apoptotic cell secrete high levels of at least one anti-apoptotic protein, including but not limited to, STC-1, BCL-2, XIAP, Survivin, and Bcl-2XL. Methods of transfecting antiapoptotic genes into MSC have been previously described which can be applied to the current invention, said antiapoptotic genes that can be utilized for practice of the invention, in a nonlimiting way, include GATA-4 [114], FGF-2 [115], bcl-2 [108, 116], and HO-1 [117]. Based upon the disclosure provided herein, MSC can be obtained from any source. The MSC may be autologous with respect to the recipient (obtained from the same host) or allogeneic with respect to the recipient. In addition, the MSC may be xenogeneic to the recipient (obtained from an animal of a different species). In one embodiment of the invention MSC are pretreated with agents to induce expression of antiapoptotic genes, one example is pretreatment with exendin-4 as previously described [118]. In a further non-limiting embodiment, MSC used in the present invention can be isolated, from the bone marrow of any species of mammal, including but not limited to, human, mouse, rat, ape, gibbon, bovine. In a non-limiting embodiment, the MSC are isolated from a human, a mouse, or a rat. In another non-limiting embodiment, the MSC are isolated from a human.

Based upon the present disclosure, MSC can be isolated and expanded in culture in vitro to obtain sufficient numbers of cells for use in the methods described herein provided that the MSC are cultured in a manner that promotes contact with a tumor endothelial cell. For example, MSC can be isolated from human bone marrow and cultured in complete medium (DMEM low glucose containing 4 mM L-glutamine, 10% FBS, and 1% penicillin/streptomycin) in hanging drops or on non-adherent dishes. The invention, however, should in no way be construed to be limited to any one method of isolating and/or to any culturing medium. Rather, any method of isolating and any culturing medium should be construed to be included in the present invention provided that the MSC are cultured in a manner that provides MSC to express increased amounts of at least one anti-apoptotic protein. Culture conditions for growth of clinical grade MSC have been described in the literature and are incorporated by reference [119-152].

### REFERENCES

1. Balamayooran, T., G. Balamayooran, and S. Jeyaseelan, Review: Toll-like receptors and NOD-like receptors in pulmonary antibacterial immunity. Innate Immun, 2010. 16(3): p. 201-10.

2. Oppeltz, R. F., et al., Burn-induced alterations in toll-like receptor-mediated responses by bronchoalveolar lavage cells. Cytokine, 2011. 55(3): p. 396-401.

3. Dolinay, T., et al., Inflammasome-regulated cytokines are critical mediators of acute lung injury. Am J Respir Crit Care Med, 2012. 185(11): p. 1225-34.

4. de Pablo, R., et al., Role of circulating soluble chemokines in septic shock. Med Intensiva, 2013. 37(8): p. 510-8.

5. Parikh, S. M., Dysregulation of the angiopoietin-Tie-2 axis in sepsis and ARDS. Virulence, 2013. 4(6): p. 517-24.

6. Nakahira, K., et al., Circulating mitochondrial DNA in patients in the ICU as a marker of mortality: derivation and validation. PLoS Med, 2013. 10(12): p. e1001577; discussion e1001577.

7. Tang, L., et al., Active players in resolution of shock/sepsis induced indirect lung injury: immunomodulatory effects of Tregs and PD-1. J Leukoc Biol, 2014. 96(5): p. 809-20.

8. Lefrancais, E., et al., Maladaptive role of neutrophil extracellular traps in pathogen-induced lung injury. JCI Insight, 2018. 3(3).

9. Yuan, Z., B. Bedi, and R.T. Sadikot, Bronchoalveolar Lavage Exosomes in Lipopolysaccharide-induced Septic Lung Injury. J Vis Exp, 2018(135).

10. Thompson, B. T., R. C. Chambers, and K.D. Liu, Acute Respiratory Distress Syndrome. N Engl J Med, 2017. 377(6): p. 562-572.

11. Moss, M., et al., The role of chronic alcohol abuse in the development of acute respiratory distress syndrome in adults. JAMA, 1996. 275(1): p. 50-4.

12. Moss, M., et al., The effect of chronic alcohol abuse on the incidence of ARDS and the severity of the multiple organ dysfunction syndrome in adults with septic shock: an interim and multivariate analysis. Chest, 1999. 116(1 Suppl): p. 97S-98S.

13. Guidot, D. M. and C. M. Hart, Alcohol abuse and acute lung injury: epidemiology and pathophysiology of a recently recognized association. J Investig Med, 2005. 53(5): p. 235-45.

14. Moss, M., et al., The effects of chronic alcohol abuse on pulmonary glutathione homeostasis. Am J Respir Crit Care Med, 2000. 161(2 Pt 1): p. 414-9.

15. Liang, Y., S. M. Yeligar, and L. A. Brown, Chronic-alcohol-abuse-induced oxidative stress in the development of acute respiratory distress syndrome. ScientificWorldJournal, 2012. 2012: p. 740308.

16. Burnham, E. L., et al., Elevated plasma and lung endothelial selectin levels in patients with acute respiratory distress syndrome and a history of chronic alcohol abuse. Crit Care Med, 2004. 32(3): p. 675-9.

17. Dada, L., et al., Alcohol worsens acute lung injury by inhibiting alveolar sodium transport through the adenosine A1 receptor. PLoS One, 2012. 7(1): p. e30448.

18. Kaphalia, L. and W. J. Calhoun, Alcoholic lung injury: metabolic, biochemical and immunological aspects. Toxicol Lett, 2013. 222(2): p. 171-9.

19. Al-Kofahi, M., et al., Finding the dose for hydroxychloroquine prophylaxis for COVID-19; the desperate search for effectiveness. Clin Pharmacol Ther, 2020.

20. shittu, M. O. and O. I. Afolami, Improving the efficacy of Chloroquine and Hydroxychloroquine against SARS-CoV-2 may require Zinc additives—A better synergy for future COVID-19 clinical trials. Infez Med, 2020. 28(2): p. 192-197.

21. Du, Y. X. and X. P. Chen, Favipiravir: Pharmacokinetics and Concerns About Clinical Trials for 2019-nCoV Infection. Clin Pharmacol Ther, 2020.

22. Ye, X. T., et al., Clinical efficacy of lopinavir/ritonavir in the treatment of Coronavirus disease 2019. Eur Rev Med Pharmacol Sci, 2020. 24(6): p. 3390-3396.

23. Su, B., et al., Efficacy and Tolerability of Lopinavir/Ritonavir—and Efavirenz-Based Initial Antiretroviral Therapy in HIV-1-Infected Patients in a Tertiary Care Hospital in Beijing, China. Front Pharmacol, 2019. 10: p. 1472.

24. Zhu, Z., et al., Arbidol monotherapy is superior to lopinavir/ritonavir in treating COVID-19. J Infect, 2020.

25. Nakamura, K., et al., A sporadic COVID-19 pneumonia treated with extracorporeal membrane oxygenation in Tokyo, Japan: A case report. J Infect Chemother, 2020.

26. Diurno, F., et al., Eculizumab treatment in patients with COVID-19: preliminary results from real life ASL Napoli 2 Nord experience. Eur Rev Med Pharmacol Sci, 2020. 24(7): p. 4040-4047.

27. Jacobs, R. F., et al., Elevated interleukin-1 release by human alveolar macrophages during the adult respiratory distress syndrome. Am Rev Respir Dis, 1989. 140(6): p. 1686-92.

28. Huang, X., et al., The Role of Macrophages in the Pathogenesis of ALI/ARDS. Mediators Inflamm, 2018. 2018: p. 1264913.

29. Schwartz, M. D., et al., Nuclear factor-kappa B is activated in alveolar macrophages from patients with acute respiratory distress syndrome. Crit Care Med, 1996. 24(8): p. 1285-92.

30. Carter, A. B., M. M. Monick, and G. W. Hunninghake, Lipopolysaccharide-induced NF-kappaB activation and cytokine release in human alveolar macrophages is PKC-independent and TK- and PC-PLC-dependent. Am J Respir Cell Mol Biol, 1998. 18(3): p. 384-91.

31. Moine, P., et al., NF-kappaB regulatory mechanisms in alveolar macrophages from patients with acute respiratory distress syndrome. Shock, 2000. 13(2): p. 85-91.

32. Saitoh, H., et al., Effect of antisense oligonucleotides to nuclear factor-kappaB on the survival of LPS-induced ARDS in mouse. Exp Lung Res, 2002. 28(3): p. 219-31.

33. Rosseau, S., et al., Phenotypic characterization of alveolar monocyte recruitment in acute respiratory distress syndrome. Am J Physiol Lung Cell Mol Physiol, 2000. 279(1): p. L25-35.

34. Jarrar, D., et al., Alveolar macrophage activation after trauma-hemorrhage and sepsis is dependent on NF-kappaB and MAPK/ERK mechanisms. Am J Physiol Lung Cell Mol Physiol, 2002. 283(4): p. L799-805.

35. Frank, J. A., et al., Alveolar macrophages contribute to alveolar barrier dysfunction in ventilator-induced lung injury. Am J Physiol Lung Cell Mol Physiol, 2006. 291(6): p. L1191-8.

36. Jiang, Z., et al., Depletion of circulating monocytes suppresses IL-17 and HMGB1 expression in mice with LPS-induced acute lung injury. Am J Physiol Lung Cell Mol Physiol, 2017. 312(2): p. L231-L242.

37. Wu, D. D., et al., Inhibition of Alveolar Macrophage Pyroptosis Reduces Lipopolysaccharide-induced Acute Lung Injury in Mice. Chin Med J (Engl), 2015. 128(19): p. 2638-45.

38. Rovira, I., et al., Effects of inhaled nitric oxide on pulmonary hemodynamics and gas exchange in an ovine model of ARDS. J Appl Physiol (1985), 1994. 76(1): p. 345-55.

39. Abman, S. H., et al., Acute effects of inhaled nitric oxide in children with severe hypoxemic respiratory failure. J Pediatr, 1994. 124(6): p. 881-8.

40. Samama, C. M., et al., Inhibition of platelet aggregation by inhaled nitric oxide in patients with acute respiratory distress syndrome. Anesthesiology, 1995. 83(1): p. 56-65.

41. Ferrer, R., et al., Anticoagulative effect of nitric oxide inhalation in ARDS. Intensive Care Med, 1998. 24(8): p. 837-8.

42. Lowson, S. M., et al., The response to varying concentrations of inhaled nitric oxide in patients with acute respiratory distress syndrome. Anesth Analg, 1996. 82(3): p. 574-81.

43. Dellinger, R. P., et al., Effects of inhaled nitric oxide in patients with acute respiratory distress syndrome: results of a randomized phase II trial. Inhaled Nitric Oxide in ARDS Study Group. Crit Care Med, 1998. 26(1): p. 15-23.

44. Troncy, E., et al., Inhaled nitric oxide in acute respiratory distress syndrome: a pilot randomized controlled study. Am J Respir Crit Care Med, 1998. 157(5 Pt 1): p. 1483-8.

45. Jacobs, B. R., et al., Aerosolized soluble nitric oxide donor improves oxygenation and pulmonary hypertension in acute lung injury. Am J Respir Crit Care Med, 1998. 158(5 Pt 1): p. 1536-42.

46. lotti, G. A., et al., Acute effects of inhaled nitric oxide in adult respiratory distress syndrome. Eur Respir J, 1998. 12(5): p. 1164-71.

47. Tang, S. F., M. C. Sherwood, and O. I. Miller, Randomised trial of three doses of inhaled nitric oxide in acute respiratory distress syndrome. Arch Dis Child, 1998. 79(5): p. 415-8.

48. Papazian, L., et al., Inhaled nitric oxide and vasoconstrictors in acute respiratory distress syndrome. Am J Respir Crit Care Med, 1999. 160(2): p. 473-9.

49. Sheridan, R. L., et al., Low-dose inhaled nitric oxide in acutely burned children with profound respiratory failure. Surgery, 1999. 126(5): p. 856-62.

50. Dupont, H., et al., Short-term effect of inhaled nitric oxide and prone positioning on gas exchange in patients with severe acute respiratory distress syndrome. Crit Care Med, 2000. 28(2): p. 304-8.

51. Gerlach, H., et al., Dose-response characteristics during long-term inhalation of nitric oxide in patients with severe acute respiratory distress syndrome: a prospective, randomized, controlled study. Am J Respir Crit Care Med, 2003. 167(7): p. 1008-15.

52. Taylor, R. W., et al., Low-dose inhaled nitric oxide in patients with acute lung injury: a randomized controlled trial. JAMA, 2004. 291(13): p. 1603-9.

53. Hsu, C. W., et al., The initial response to inhaled nitric oxide treatment for intensive care unit patients with acute respiratory distress syndrome. Respiration, 2008. 75(3): p. 288-95.

54. McIntyre, R. C., Jr., et al., Inhaled nitric oxide variably improves oxygenation and pulmonary hypertension in patients with acute respiratory distress syndrome. J Trauma, 1995. 39(3): p. 418-25.

55. Rossaint, R., et al., Efficacy of inhaled nitric oxide in patients with severe ARDS. Chest, 1995. 107(4): p. 1107-15.

56. Biffl, W. L., et al., Nitric oxide reduces endothelial expression of intercellular adhesion molecule (ICAM)-1. J Surg Res, 1996. 63(1): p. 328-32.

57. Wu, H. S., et al., Effect of nitric oxide on toll-like receptor 2 and 4 gene expression in rats with acute lung injury complicated by acute hemorrhage necrotizing pancreatitis. Hepatobiliary Pancreat Dis Int, 2005. 4(4): p. 609-13.

58. Ader, F., et al., Inhaled nitric oxide increases endothelial permeability in Pseudomonas aeruginosa pneumonia. Intensive Care Med, 2007. 33(3): p. 503-10.

59. Qi, Y., et al., Inhaled NO contributes to lung repair in piglets with acute respiratory distress syndrome via increasing circulating endothelial progenitor cells. PLoS One, 2012. 7(3): p. e33859.

60. Afshari, A., et al., Inhaled nitric oxide for acute respiratory distress syndrome (ARDS) and acute lung injury in children and adults. Cochrane Database Syst Rev, 2010(7): p. CD002787.

61. Angus, D. C., et al., Healthcare costs and long-term outcomes after acute respiratory distress syndrome: A phase III trial of inhaled nitric oxide. Crit Care Med, 2006. 34(12): p. 2883-90.

62. Gebistorf, F., et al., Inhaled nitric oxide for acute respiratory distress syndrome (ARDS) in children and adults. Cochrane Database Syst Rev, 2016(6): p. CD002787.

63. Cullen, S. C. and E. G. Gross, The anesthetic properties of xenon in animals and human beings, with additional observations on krypton. Science, 1951. 113(2942): p. 580-2.

64. Dickinson, R. and N. P. Franks, Bench-to-bedside review: Molecular pharmacology and clinical use of inert gases in anesthesia and neuroprotection. Crit Care. 14(4): p. 229.

65. Fahlenkamp, A. V., R. Rossaint, and M. Coburn, [Neuroprotection by noble gases: New developments and insights]. Anaesthesist, 2015. 64(11): p. 855-8.

66. Peng, T., et al., Therapeutic time window and dose dependence of xenon delivered via echogenic liposomes for neuroprotection in stroke. CNS Neurosci Ther, 2013. 19(10): p. 773-84.

67. Deng, J., et al., Neuroprotective gases-fantasy or reality for clinical use? Prog Neurobiol, 2014. 115: p. 210-45.

68. Dixon, B. J., et al., Neuroprotective Strategies after Neonatal Hypoxic Ischemic Encephalopathy. Int J Mol Sci, 2015. 16(9): p. 22368-401.

69. Sutherland, B. A., et al., Inhalation gases or gaseous mediators as neuroprotectants for cerebral ischaemia. Curr Drug Targets, 2013. 14(1): p. 56-73.

70. Van Pham, P., et al., Isolation and proliferation of umbilical cord tissue derived mesenchymal stem cells for clinical applications. Cell Tissue Bank, 2015.

71. Fazzina, R., et al., A new standardized clinical-grade protocol for banking human umbilical cord tissue cells. Transfusion, 2015. 55(12): p. 2864-73.

72. Bieback, K., Platelet lysate as replacement for fetal bovine serum in mesenchymal stromal cell cultures. Transfus Med Hemother, 2013. 40(5): p. 326-35.

73. Stanko, P., et al., Comparison of human mesenchymal stem cells derived from dental pulp, bone marrow, adipose tissue, and umbilical cord tissue by gene expression. Biomed Pap Med Fac Univ Palacky Olomouc Czech Repub, 2014. 158(3): p. 373-7.

74. Schira, J., et al., Significant clinical, neuropathological and behavioural recovery from acute spinal cord trauma by transplantation of a well-defined somatic stem cell from human umbilical cord blood. Brain, 2012. 135(Pt 2): p. 431-46.

75. Hartmann, I., et al., Umbilical cord tissue-derived mesenchymal stem cells grow best under GMP-compliant culture conditions and maintain their phenotypic and functional properties. J Immunol Methods, 2010. 363(1): p. 80-9.

76. Friedman, R., et al., Umbilical cord mesenchymal stem cells: adjuvants for human cell transplantation. Biol Blood Marrow Transplant, 2007. 13(12): p. 1477-86.

77. Zhang, X. Y., et al., Lentiviral vectors for sustained transgene expression in human bone marrow-derived stromal cells. Mol Ther, 2002. 5(5 Pt 1): p. 555-65.

78. Kyriakou, C. A., et al., Human mesenchymal stem cells (hMSCs) expressing truncated soluble vascular endothelial growth factor receptor (tsFlk-1) following lentiviral-mediated gene transfer inhibit growth of Burkitt's lymphoma in a murine model. J Gene Med, 2006. 8(3): p. 253-64.

79. Worsham, D. N., et al., In vivo gene transfer into adult stem cells in unconditioned mice by in situ delivery of a lentiviral vector. Mol Ther, 2006. 14(4): p. 514-24.

80. Rabin, N., et al., A new xenograft model of myeloma bone disease demonstrating the efficacy of human mesenchymal stem cells expressing osteoprotegerin by lentiviral gene transfer. Leukemia, 2007. 21(10): p. 2181-91.

81. Kallifatidis, G., et al., Improved lentiviral transduction of human mesenchymal stem cells for therapeutic intervention in pancreatic cancer. Cancer Gene Ther, 2008. 15(4): p. 231-40.

82. Meyerrose, T. E., et al., Lentiviral-transduced human mesenchymal stem cells persistently express therapeutic levels of enzyme in a xenotransplantation model of human disease. Stem Cells, 2008. 26(7): p. 1713-22.

83. McGinley, L., et al., Lentiviral vector mediated modification of mesenchymal stem cells & enhanced survival in an in vitro model of ischaemia. Stem Cell Res Ther, 2011. 2(2): p. 12.

84. Liang, X., et al., Human bone marrow mesenchymal stem cells expressing SDF-1 promote hematopoietic stem cell function of human mobilised peripheral blood CD34+ cells in vivo and in vitro. Int J Radiat Biol, 2010. 86(3): p. 230-7.

85. Glavaski-Joksimovic, A., et al., Glial cell line-derived neurotrophic factor-secreting genetically modified human bone marrow-derived mesenchymal stem cells promote recovery in a rat model of Parkinson's disease. J Neurosci Res, 2010. 88(12): p. 2669-81.

86. Liu, A. M., et al., Umbilical cord-derived mesenchymal stem cells with forced expression of hepatocyte growth factor enhance remyelination and functional recovery in a rat intracerebral hemorrhage model. Neurosurgery, 2010. 67(2): p. 357-65; discussion 365-6.

87. Yu, Y. S., et al., AKT-modified autologous intracoronary mesenchymal stem cells prevent remodeling and repair in swine infarcted myocardium. Chin Med J (Engl), 2010. 123(13): p. 1702-8.

88. Mueller, L. P., et al., TRAIL-transduced multipotent mesenchymal stromal cells (TRAIL-MSC) overcome TRAIL resistance in selected CRC cell lines in vitro and in vivo. Cancer Gene Ther, 2011. 18(4): p. 229-39.

89. Yan, C., et al., Suppression of orthotopically implanted hepatocarcinoma in mice by umbilical cord-derived mesenchymal stem cells with sTRAIL gene expression driven by AFP promoter. Biomaterials, 2014. 35(9): p. 3035-43.

90. Deng, Q., et al., TRAIL-secreting mesenchymal stem cells promote apoptosis in heat-shock-treated liver cancer cells and inhibit tumor growth in nude mice. Gene Ther, 2014. 21(3): p. 317-27.

91. Sage, E. K., et al., Systemic but not topical TRAIL-expressing mesenchymal stem cells reduce tumour growth in malignant mesothelioma. Thorax, 2014. 69(7): p. 638-47.

92. Lian, W. S., et al., In vivo therapy of myocardial infarction with mesenchymal stem cells modified with prostaglandin I synthase gene improves cardiac performance in mice. Life Sci, 2011. 88(9-10): p. 455-64.

93. Maijenburg, M. W., et al., Nuclear receptors Nur77 and Nurr1 modulate mesenchymal stromal cell migration. Stem Cells Dev, 2012. 21(2): p. 228-38.

94. Harper, M. M., et al., Transplantation of BDNF-secreting mesenchymal stem cells provides neuroprotection in chronically hypertensive rat eyes. Invest Ophthalmol Vis Sci, 2011. 52(7): p. 4506-15.

95. Zou, D., et al., In vitro study of enhanced osteogenesis induced by HIF-1alpha-transduced bone marrow stem cells. Cell Prolif, 2011. 44(3): p. 234-43.

96. Saito, S., et al., Mesenchymal stem cells stably transduced with a dominant-negative inhibitor of CCL2 greatly attenuate bleomycin-induced lung damage. Am J Pathol, 2011. 179(3): p. 1088-94.

97. Seo, K. W., et al., Anti-tumor effects of canine adipose tissue-derived mesenchymal stromal cell-based interferon-beta gene therapy and cisplatin in a mouse melanoma model. Cytotherapy, 2011. 13(8): p. 944-55.

98. Yang, H. M., et al., Enhancement of the immunosuppressive effect of human adipose tissue-derived mesenchymal stromal cells through HLA-G1 expression. Cytotherapy, 2012. 14(1): p. 70-9.

99. Liang, X. J., et al., Differentiation of human umbilical cord mesenchymal stem cells into hepatocyte-like cells by hTERT gene transfection in vitro. Cell Biol Int, 2012. 36(2): p. 215-21.

100. Fei, S., et al., The antitumor effect of mesenchymal stem cells transduced with a lentiviral vector expressing cytosine deaminase in a rat glioma model. J Cancer Res Clin Oncol, 2012. 138(2): p. 347-57.

101. Jaganathan, B. G. and D. Bonnet, Human mesenchymal stromal cells senesce with exogenous OCT4. Cytotherapy, 2012. 14(9): p. 1054-63.

102. Han, S. H., et al., Effect of ectopic OCT4 expression on canine adipose tissue-derived mesenchymal stem cell proliferation. Cell Biol Int, 2014. 38(10): p. 1163-73.

103. Shangguan, L., et al., Inhibition of TGF-beta/Smad signaling by BAMBI blocks differentiation of human mesenchymal stem cells to carcinoma-associated fibroblasts and abolishes their protumor effects. Stem Cells, 2012. 30(12): p. 2810-9.

104. Kearns-Jonker, M., et al., Genetically Engineered Mesenchymal Stem Cells Influence Gene Expression in Donor Cardiomyocytes and the Recipient Heart. J Stem Cell Res Ther, 2012. S1.

105. Ma, G. L., et al., [Study of inhibiting and killing effects of transgenic LIGHT human umbilical cord blood mesenchymal stem cells on stomach cancer]. Zhonghua Wei Chang Wai Ke Za Zhi, 2012. 15(11): p. 1178-81.

106. Huang, F., et al., Mesenchymal stem cells modified with miR-126 release angiogenic factors and activate Notch ligand Delta-like-4, enhancing ischemic angiogenesis and cell survival. Int J Mol Med, 2013. 31(2): p. 484-92.

107. Huang, F., et al., Overexpression of miR-126 promotes the differentiation of mesenchymal stem cells toward endothelial cells via activation of PI3K/Akt and MAPK/ERK pathways and release of paracrine factors. Biol Chem, 2013. 394(9): p. 1223-33.

108. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.

109. Madonna, R., et al., Transplantation of mesenchymal cells rejuvenated by the overexpression of telomerase and myocardin promotes revascularization and tissue repair in a murine model of hindlimb ischemia. Circ Res, 2013. 113(7): p. 902-14.

110. Zang, Y., et al., [Influence of CXCR4 overexpressed mesenchymal stem cells on hematopoietic recovery of irradiated mice]. Zhongguo Shi Yan Xue Ye Xue Za Zhi, 2013. 21(5): p. 1261-5.

111. Cao, Z., et al., Protective effects of mesenchymal stem cells with CXCR4 up-regulation in a rat renal transplantation model. PLoS One, 2013. 8(12): p. e82949.

112. Liu, S., et al., Overexpression of Wnt11 promotes chondrogenic differentiation of bone marrow-derived mesenchymal stem cells in synergism with TGF-beta. Mol Cell Biochem, 2014. 390(1-2): p. 123-31.

113. Zhu, Y., et al., Mesenchymal stem cell-based NK4 gene therapy in nude mice bearing gastric cancer xenografts. Drug Des Devel Ther, 2014. 8: p. 2449-62.

114. Yu, B., et al., Enhanced mesenchymal stem cell survival induced by GATA-4 overexpression is partially mediated by regulation of the miR-15 family. Int J Biochem Cell Biol, 2013. 45(12): p. 2724-35.

115. Xu, W., et al., Basic fibroblast growth factor expression is implicated in mesenchymal stem cells response to light-induced retinal injury. Cell Mol Neurobiol, 2013. 33(8): p. 1171-9.

116. Li, W., et al., Bcl-2 engineered MSCs inhibited apoptosis and improved heart function. Stem Cells, 2007. 25(8): p. 2118-27.

117. Tsubokawa, T., et al., Impact of anti-apoptotic and anti-oxidative effects of bone marrow mesenchymal stem cells with transient overexpression of heme oxygenase-1 on myocardial ischemia. Am J Physiol Heart Circ Physiol, 2010. 298(5): p. H1320-9.

118. Zhou, H., et al., Exendin-4 protects adipose-derived mesenchymal stem cells from apoptosis induced by hydrogen peroxide through the PI3K/Akt-Sfrp2 pathways. Free Radic Biol Med, 2014. 77: p. 363-75.

119. Le Blanc, K., et al., Treatment of severe acute graft-versus-host disease with third party haploidentical mesenchymal stem cells. Lancet, 2004. 363(9419): p. 1439-41.

120. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11(5): p. 389-98.

121. Bernardo, M. E., et al., Optimization of in vitro expansion of human multipotent mesenchymal stromal cells for cell-therapy approaches: further insights in the search for a fetal calf serum substitute. J Cell Physiol, 2007. 211(1): p. 121-30.

122. Reinisch, A., et al., Humanized system to propagate cord blood-derived multipotent mesenchymal stromal cells for clinical application. Regen Med, 2007. 2(4): p. 371-82.

123. Capelli, C., et al., Human platelet lysate allows expansion and clinical grade production of mesenchymal stromal cells from small samples of bone marrow aspirates or marrow filter washouts. Bone Marrow Transplant, 2007. 40(8): p. 785-91.

124. Lataillade, J. J., et al., New approach to radiation burn treatment by dosimetry-guided surgery combined with autologous mesenchymal stem cell therapy. Regen Med, 2007. 2(5): p. 785-94.

125. Seshareddy, K., D. Troyer, and M. L. Weiss, Method to isolate mesenchymal-like cells from Wharton's Jelly of umbilical cord. Methods Cell Biol, 2008. 86: p. 101-19.

126. Sensebe, L., Clinical grade production of mesenchymal stem cells. Biomed Mater Eng, 2008. 18(1 Suppl): p. S3-10.

127. Sotiropoulou, P. A., S. A. Perez, and M. Papamichail, Clinical grade expansion of human bone marrow mesenchymal stem cells. Methods Mol Biol, 2007. 407: p. 245-63.

128. Shetty, P., et al., Clinical grade mesenchymal stem cells transdifferentiated under xenofree conditions alleviates motor deficiencies in a rat model of Parkinson's disease. Cell Biol Int, 2009. 33(8): p. 830-8.

129. Zhang, X., et al., Cotransplantation of HLA-identical mesenchymal stem cells and hematopoietic stem cells in Chinese patients with hematologic diseases. Int J Lab Hematol, 2010. 32(2): p. 256-64.

130. Arrigoni, E., et al., Isolation, characterization and osteogenic differentiation of adipose-derived stem cells: from small to large animal models. Cell Tissue Res, 2009. 338(3): p. 401-11.

131. Grisendi, G., et al., GMP-manufactured density gradient media for optimized mesenchymal stromal/stem cell isolation and expansion. Cytotherapy, 2010. 12(4): p. 466-77.

132. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.

133. Sensebe, L., P. Bourin, and K. Tarte, Good manufacturing practices production of mesenchymal stem/stromal cells. Hum Gene Ther, 2011. 22(1): p. 19-26.

134. Capelli, C., et al., Minimally manipulated whole human umbilical cord is a rich source of clinical-grade human mesenchymal stromal cells expanded in human platelet lysate. Cytotherapy, 2011. 13(7): p. 786-801.

135. Ilic, N., et al., Manufacture of clinical grade human placenta-derived multipotent mesenchymal stromal cells. Methods Mol Biol, 2011. 698: p. 89-106.

136. Santos, F., et al., Toward a clinical-grade expansion of mesenchymal stem cells from human sources: a microcarrier-based culture system under xeno-free conditions. Tissue Eng Part C Methods, 2011. 17(12): p. 1201-10.

137. Timmins, N. E., et al., Closed system isolation and scalable expansion of human placental mesenchymal stem cells. Biotechnol Bioeng, 2012. 109(7): p. 1817-26.

138. Warnke, P. H., et al., A clinically-feasible protocol for using human platelet lysate and mesenchymal stem cells in regenerative therapies. J Craniomaxillofac Surg, 2013. 41(2): p. 153-61.

139. Fekete, N., et al., GMP-compliant isolation and large-scale expansion of bone marrow-derived MSC. PLoS One, 2012. 7(8): p. e43255.

140. Hanley, P. J., et al., Manufacturing mesenchymal stromal cells for phase I clinical trials. Cytotherapy, 2013. 15(4): p. 416-22.

141. Trojahn Kolle, S. F., et al., Pooled human platelet lysate versus fetal bovine serum-investigating the proliferation rate, chromosome stability and angiogenic potential of human adipose tissue-derived stem cells intended for clinical use. Cytotherapy, 2013. 15(9): p. 1086-97.

142. Veronesi, E., et al., Transportation conditions for prompt use of ex vivo expanded and freshly harvested clinical-grade bone marrow mesenchymal stromal/stem cells for bone regeneration. Tissue Eng Part C Methods, 2014. 20(3): p. 239-51.

143. Dolley-Sonneville, P. J., L. E. Romeo, and Z. K. Melkoumian, Synthetic surface for expansion of human mesenchymal stem cells in xeno-free, chemically defined culture conditions. PLoS One, 2013. 8(8): p. e70263.

144. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.

145. Martins, J. P., et al., Towards an advanced therapy medicinal product based on mesenchymal stromal cells isolated from the umbilical cord tissue: quality and safety data. Stem Cell Res Ther, 2014. 5(1): p. 9.

146. ludicone, P., et al., Pathogen-free, plasma-poor platelet lysate and expansion of human mesenchymal stem cells. J Transl Med, 2014. 12: p. 28.

147. Skrahin, A., et al., Autologous mesenchymal stromal cell infusion as adjunct treatment in patients with multidrug and extensively drug-resistant tuberculosis: an open-label phase 1 safety trial. Lancet Respir Med, 2014. 2(2): p. 108-22.

148. Ikebe, C. and K. Suzuki, Mesenchymal stem cells for regenerative therapy: optimization of cell preparation protocols. Biomed Res Int, 2014. 2014: p. 951512.

149. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.

150. Swamynathan, P., et al., Are serum-free and xeno-free culture conditions ideal for large scale clinical grade expansion of Wharton's jelly derived mesenchymal stem cells? A comparative study. Stem Cell Res Ther, 2014. 5(4): p. 88.

151. Vaes, B., et al., Culturing protocols for human multipotent adult stem cells. Methods Mol Biol, 2015. 1235: p. 49-58.

152. Devito, L., et al., Wharton's jelly mesenchymal stromal/stem cells derived under chemically defined animal product-free low oxygen conditions are rich in MSCA-1(+)subpopulation. Regen Med, 2014. 9(6): p. 723-32.

1. A method of treating pulmonary inflammation comprising administering to a patient in need of therapy a therapeutic gas composition at a concentration and frequency needed to reduce said pulmonary inflammation.

2. The method of claim 1, wherein said pulmonary inflammation is acute respiratory distress syndrome possessing enhanced levels of inflammatory cytokines in the lung as compared to an age-matched control lung.

3. The method of claim 2, wherein said inflammatory cytokines are selected from a group comprising of: a) interleukin 1; b) interleukin 6; c) interleukin 8; d) interleukin-11; e) interleukin 12; f) interleukin 15; g) interleukin 17; h) interleukin 18; i) interleukin 33; j) TNF-alpha; k) HMGB1; and 1) MCP-1.

4. The method of claim 1, wherein said therapeutic gases comprise of a mixture of one or more gases: a) carbon monoxide; b) argon; c) helium; c) radon; d) krypton; e) neon; f) xenon; g) hydrogen; h) hydrogen sulfide; i) ozone and j) nitric oxide.

5. The method of claim 4, wherein said therapeutic gases comprise a proportion by volume of 20 to 70% of therapeutic gas and the rest oxygen and/or air.

6. The method of claim 5, wherein said proportion of xenon is between 22 and 60% by volume to oxygen.

7. The method of claim 6, wherein said proportion of xenon is between 25 and 60% by volume to oxygen.

8. The method of claim 4, wherein said therapeutic gas consists of a) oxygen and xenon or b) air and xenon.

9. The method of claim 4, wherein said therapeutic gas mixture also contains nitrogen, helium, Nitric Oxide, krypton, argon, hydrogen sulfide or neon.

10. The method of claim 1, wherein said therapeutic gas mixture contains a proportion by volume of oxygen of between 15 and 25%.

11. The method of claim 1, wherein said therapeutic gas mixture is supplied for inhalation from a pressurized container at a pressure greater than 2 bar.

12. The method of claim 1, wherein said therapeutic gas mixture is administered intranasally.

13. The method of claim 1, wherein said therapeutic gas mixture is administered through the use of a hyperbaric chamber.

14. The method of claim 13, wherein said hyperbaric chamber is pressurized to a pressure of no more than 3 atm (0.3 MPa).

15. The method of claim 14, wherein a therapeutic gas mixture is administered to the patient while the patient is in the hyperbaric environment.

16. The method of claim 1 wherein said therapeutic gas mixture is administered by inhalation or simulated inhalation.

17. The method of claim 1, wherein said therapeutic gas mixture is xenon, helium, or a mixture of xenon and helium.

18. The method of claim 1, wherein the therapeutic gas mixture is xenon or a mixture of xenon and helium, and the partial pressure of xenon is no more than about 0.8 atm (0.08 MPa).

19. The method of claim 1, wherein said therapeutic gas mixture is administered mixed with air, the air partial pressure being about 1 atm (0.1 MPa).

20. The method of claim 1, wherein said therapeutic gas mixture is administered as part of a gas mixture comprising oxygen, the nitrogen partial pressure in the mixture being equal to or less than about 0.8 atm (0.08 MPa).
